(19) United States
(12) Patent Application Publication
(10) Pub. No.: US 2007/0031360 A1
(43) Pub. Date: Feb. 8, 2007

(54) Stripless Depilatory Compositions

(75) Inventor: Shyam K Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 11/161,575

(22) Filed: Aug. 8, 2005

(51) Int. Cl. A61K 8/81

(52) U.S. Cl. 424/70.11

(57) ABSTRACT

A depilatory composition that can be applied in liquid form on the surface of the skin, and which is removable by peeling away from the surface of the skin after setting to a pliable film, said composition consisting essentially of (i) a pliable film forming agent, (ii) a hair binding agent, and (iii) a hydroxylic matrix forming agent, and, wherein the relative amounts of said pliable film forming agent, said hair binding agent, and said hydroxylic matrix forming agent are such that said composition upon first heating to a liquid state, then applying to skin transforms to a pliable film, which is capable of catching hairs without adhering to the skin and being removable by peeling away from the skin along with the hairs caught in the pliable film.

Depilatory compositions formed of viscoelastic materials are well known. The viscoelastic materials may in certain embodiments be rosin-based. In other embodiments they may be sugar-based. A tackifier may be included to make them sticky. These aspects have been discussed by the present inventor in two recent disclosures (U.S. patent application Ser. Nos. 20040175340 and 20040180014), both of which are not related to the present invention.

In some products the depilatory compositions may be supplied in the form of strips, retained between cellophane sheets. The cellophane sheets may have coatings of polyvinyl chloride, which acts as a barrier preventing the composition, or components of it, from migrating through the sheets; and also having the correct adhesive properties for use. In use, the user peels away one of the cellophane sheets, presses the depilatory strip firmly onto the area to be epilated, then pulls one end of the remaining cellophane sheet sharply away from the area in a direction opposite to hair growth angle. The hairs trapped in the composition are removed from the treated area along with, optimally, all of the composition, still attached to the remaining backing strip.

In an alternative approach a composition may be warmed, and then applied to the skin by means of a spatula or other applicator. Strips of fabric are then applied so that they adhere to the depilatory composition. The strips are then pulled sharply to remove the depilatory material, and hair, from the skin. U.S. patent application nos. 20050085828, 20050079189, and 20050019292 disclose such compositions.

In both approaches the viscoelastic properties of the compositions are important. However this is particularly so in the case of the compositions supplied as strips, since these are applied to the skin at ambient temperature. At ambient temperature the compositions should be soft and pliable, such that they mould closely to the body shape. On the other hand they must not be so soft that they flow prior to use. When they are in place on the body and the user pulls the remaining backing strip, applying a high frequency strain rate to the compositions, their elastic properties should predominate over their viscous properties.

There is a particular problem with known compositions supplied in the form of strips, in meeting one of the requirements described in the previous paragraph. It is that under warm ambient conditions the compositions may flow, and leak out from between the sheets. One approach to counter this has been to supply strips with considerably over-sized cellophane sheets. However, clearly, this approach is inadequate as a solution to a situation where there might be a substantial flow of a composition. It is also inefficient in terms of materials and transportation, and undesirable from a marketing perspective, in that consumers perceive that such a product is of poor value.

Depilatory compositions, particularly compositions supplied in the form of strips, may include non-aqueous based aliphatic or aromatic rosin, for example a rosinous material. Suitably, such compositions may exhibit strong adhesive properties, which render them suitable to trap and remove hairs from a treated area. However, there are particular disadvantages associated with known non-aqueous based aliphatic or aromatic resins. Notably, as a consequence of the strong adhesive properties of these resins, skin as well as hairs may be removed from the treated area, thereby causing discomfort and irritation for the user. Suitably, it may be undesirable for users having sensitive skin to use such compositions and/or to use such compositions on sensitive parts of the body. Moreover, such resins may bond strongly with the skin and the resin may separate from the strip when the strip is pulled from the treated area, thereby leaving a deposit of the resin on the skin. It is typically difficult to remove such a resinous deposit merely by rinsing the treated area with water, because of the non-aqueous characteristics of the resin. Suitably, in order to remove the resin from the treated area, it is typically necessary to wipe the treated area with a cloth impregnated with an organic solvent mixture that dissolves and/or loosens the resin. Such a procedure not only involves applying an organic solvent to the skin which may cause further discomfort and/or irritation, but also it is inconvenient and creates unnecessary additional expense for the user.

In an attempt to overcome the aforementioned problems associated with depilatory compositions including non-aqueous based resins, attention has focused on compositions that include aqueous based materials, such as sugar-based materials. Suitably, a deposit of an aqueous based composition left on a treated area following removal of a strip may be rinsed more easily from the area using water compared to a non-aqueous based epilatory composition, thereby reducing or negating the need to use a cloth impregnated with an organic solvent. However, aqueous based materials typically exhibit lower adhesive properties than non-aqueous based resins. Suitably, aqueous based materials may be less effective at removing hairs from a treated area than non-aqueous based resins. Also, aqueous based compositions tend to lose water upon repeated opening and closing of containers in which such compositions are stored and used. This causes the crystallization of aqueous soluble ingredients in such compositions, which renders such compositions less effective, or even ineffective, in removing hair with their progressive use.

There exists a third approach, whereby a composition is applied to skin in a liquid state, and which upon cooling to room temperature transforms into a plastic like material that can be peeled to remove hair entrapped in it. This is further disclosed in U.S. Pat. Nos. 5,154,919 and 4,734,099. Additionally, certain commercial products are marketed with stripless depilatory claims, such as Surgiwax Microwave Hair Removal (also called Brazilian Wax), which contains Rosin, Glycerol Rosinate, Beeswax, Methyl Hydrogenated Rosinate, Glycerin, Paraffin, Soybean oil, Ethylhexyl Palmitate, Methyl Paraben, Propyl Paraben, Titanium Dioxide, Red 17, and Blue 1. Another such product, Surgiwax Microwave for Body, contains Rosin, Beeswax, Glycerin, Methyl Paraben, and Propyl Paraben. Exact percentages of these compositions are unknown, although it is assumed that such ingredients are present in their decreasing order of preponderance. These compositions are applied by first heating to a liquid state, then applying to skin, which transforms them to a plasticized film, which is capable of catching hairs and being removable by peeling away from the skin along with the hairs caught in the plasticized film. However, it may be advantageous if such compositions can also allow the use of a strip to permit a dual removal system. Also, it would offer additional advantage if such compositions can also be pre-coated on a strip to offer convenience to certain consumers.

U.S. Pat. No. 4,734,099 (Cyprien) discloses a process for the removal of unwanted hair from predetermined skin area, comprising the steps of (a) applying to said skin area an effective depilating amount of a coating of a curable organopolysiloxane composition comprising (i) a diorganopolysiloxane polymer containing at least 2 vinyl radicals bonded to silicon atoms per molecule, (ii) a diorganopolysiloxane polymer containing at least 3 hydrogen atoms bonded to silicon atoms per molecule, (iii) a filler, and (iv) a metal catalyst wherein components (i)-(iv) are present in amounts sufficient to produce a composition which is effective for the removal of hair; (b) permitting said organopolysiloxane composition to cross link into a silicone elastomer and embed unwanted hair therein; and (c) stripping the silicone elastomer and embedded hair from said skin area, whereby said skin area is essentially depilated. This method is too complex, the silicon materials are not easily available, and silicone monomers utilized have low boiling points, which causes their rapid evaporation before the completion of cross linking process thus resulting in variable results in depilation.

The disclosure in U.S. Pat. No. 5,154,919 (Garets) is of most significance. The compositions disclosed in this patent do perform better than the traditional compositions. Garets utilizes a combination of an elastomer (5 to 15%), a resinous material (40 to 80%), and a softener (10 to 20%). Presumably, the elastomer forms a film, while the resinous material is responsible for trapping and removing the hair, and softener provides the elasticity to the film. However, Garets compositions do not peel off as a single piece. Upon application to skin the film that is formed is still too brittle, which breaks into small pieces upon pulling to remove hair. It would be obvious to any one skilled in the art that this problem could be solved easily by increasing the amount of elastomer in Garets compositions. In order to increase the amount of elastomer in such compositions, the amounts of resinous material and/or softener must be reduced. This causes the problem, as the hair removal efficiency of such modified compositions is greatly reduced. This is because if the resinous material is reduced that causes the obvious reduction of hair binding ability of such compositions. If the softener is reduced, then the film formed on the skin becomes too brittle. In addition, the formation of Garets compositions requires heating the ingredients from 90 to 160C. This high heat is necessary because the elastomeric material is not soluble in the resinous material, as stated by Garets. This causes a serious limitation in the addition of other beneficial ingredients, such as colorants, fragrances, and skin emollients, to such compositions. Overall, the concept of a stripless hair removal composition, as disclosed by Garets, merits attention relative to further improvement.

In the design of an optimally performing stripless composition for hair removal, it would be advantageous if the following benefits can be combined: (1) No need for an extra strip (the composition itself transforms into a strip), (2) Easy removal of strip (formed by the composition itself) in a single attempt, (3) Complete removal of hair in a single application, (4) None (or minimal) residue for clean-up, (5) Least adhesion to skin, and (6) Low temperature (temperatures less than 100C.) processability.

### SUMMARY OF THE INVENTION.

It has now been discovered that a film forming (or, elastomeric) material can be combined in higher amounts (to provide improved film forming benefits) without reducing the amount of a hair-binding agent. Instead of using a softener, as in Garets disclosure, a matrix-forming agent is used. The function of matrix forming agent is to solubilize film-forming agent into hair binding agent via a matrix formation. It is like forming a fabric sheet, which has warp and weft or woof components. While Garets uses a softener, which are a fat, an oil, and a wax, the present invention has found that a Hydroxy or Polyhydroxy material, such as various glycols and polyglycols, surprisingly and unexpectedly, act as excellent matrix forming agents. It is not known at present how or why such hydroxylic materials form a matrix in combination with a film forming agent and a hair-binding agent. It is well known that hydroxy and Polyhydroxy compounds have the ability to form hydrogen bonds with electron donating groups, which can provide both flexibility and strength to film formed by the film forming agent in the present invention. Although most of film forming agents utilized in the present invention do possess electron donating atoms (such as oxygen), the exact mechanism of such, if any, hydrogen bonds formed by the hydroxylic matrix forming agents in the present invention is not known at this time. Nevertheless, such a lack of knowledge should not detract from the commercial or practical utility of such compositions in efficient hair removal applications.

The present invention is comprised of a stripless depilatory composition that can be applied in liquid form on the surface of the skin, and which is removable by peeling away from the surface of the skin after setting to a pliable film, said composition consisting essentially of (i) a pliable film forming agent in 20 to 50 weight % amount, (ii) a hair binding agent in 50 to 80 weight % amount, and (iii) a hydroxylic matrix forming agent in 1 to 20 weight % amount, and, wherein the relative amounts of said pliable film forming agent, said hair binding agent, and said hydroxylic matrix forming agent are such that said composition upon first heating to a liquid state, then applying to skin transforms to a pliable film, which is capable of catching hairs without adhering to the skin and being removable by peeling away from the skin along with the hairs caught in the pliable film.

The pliable film-forming agent is selected from paraffin, wax, polyvinyl acetate, modified cellulose, carbowax, tribehenin, and Glyceryl behenate.

The hair binding agent is selected from to Wood Rosin, Rosin Esters, Modified Rosin Esters, Glyceryl Rosinate, Glyceryl Hydrogenated Rosinate, Polyethylene Glycol Rosinate, Polyethylene Glycol Hydrogenated Rosinate, Pentaerythritol Rosinate, Pentaerythritol Hydrogenated Rosinate, Polyvinyl Alcohol, Polyvinyl Acetate, Polyvinyl Esters, PVP, PVP/PVA Copolymers, Dimerized Rosin, Esters of Dimerized Rosin, Modified Wood Rosin, Esters of Modified Wood Rosin, Polyol Ester Rosinate, Polyterpene Resins, Esters of Hydrogenated Modified Rosin; (Hydrogenated Rosins covers both fully and partially hydrogenated forms), Polyethylene, Polypropylene, Polystyrene, Polyisobutylenes, EVA Resins, Block Copolymers, Polyvinyl Ethers, Polyacrylics, Polyvinyl butyral, Polyamides, Aromatic Hydrocarbon Resin, Cellulose Acetate, Ethyl Cellulose, Cellulose Acetate Butyrate, Ethyl Hydroxyethylcellulose, Nitrocellulose, Alkyl Resins, Rosin Ester Resins, Hydrocarbon resins, Natural Waxes, Shellac, Natural Rubber, Styrene-Butadiene Rubber, Nitrile Rubber, Butyl Rubber, Polychloroprene Rubber, and Chlorinated Rubber.

The hydroxylic matrix forming agent is selected from polyethylene glycol, polypropylene glycol, polybutylene glycol, carbowax derivatives, Laureth-3, Laureth-4, Polysorbates, Polysorbate Esters, Polyglyceryl Stearates, propylene glycol, methylpropanediol, butylene glycol, hexylene glycol, ethoxydiglycol, Triethyl citrate, Trimethyl citrate, Tripropyl citrate, Tributyl citrate, Trihexyl citrate, Tribenzyl citrate, Butyl Trihexyl citrate, Acetyl Triethyl Citrate, Acetyl-tri-n-butyl Citrate, Acetyl-tri-n-hexyl Citrate, n-Butyl tri-n-hexyl Citrate, Methyl lactate, Ethyl lactate, Propyl lactate, Isopropyl lactate, Butyl lactate, Benzyl lactate and combinations thereof.

It is worthy of note that the compositions of the present invention can be made at temperatures less than 100C., as further illustrated in the Examples section of the present invention. This is of special significance since all previously known hot-wax compositions based on rosinates have all required processing temperatures much above 100C. The compositions of the present invention provide far more convenient commercial manufacturing process, and a much greater number of other additives, fragrances, and compositions can also be included, since they can now be added at temperatures in the range of 50 to 55C. after mixing of the main batch is complete and batch has been cooled down to a lower temperature for the addition of any temperature sensitive ingredients or compositions.

It is important to note here that the compositions of the present invention that do not contain a hydroxylic matrix forming agent do not provide satisfactory hair removal, as further illustrated in Example 6. Also, a composition (Example 13) prepared according to U.S. Pat. No. 5,1 54,919 (Garets), did not perform as well.

### EXAMPLES

The following examples are presented to illustrate presently preferred practice thereof. As illustrations they are not intended to limit the scope of the invention. All quantities are in weight %.

### Example 1

Stripless Depilatory Composition 1.

Ingredients. (1) Glyceryl Hydrogenated Rosinate 72.0 (2) Polypropylene Glycol 5.0 (3) Paraffin 16.0 (4) Beeswax 7.0. Procedure. Mix all components and heat at 90 to 95C. to a clear liquid. Cool to room temperature.

### Example 2

Stripless Depilatory Composition 2.

Ingredients. (1) Glyceryl Hydrogenated Rosinate 50.0 (2) Paraffin 40.0 (3) Beeswax 10.0. Procedure. Mix all components and heat at 90 to 95C. to a clear liquid. Cool to room temperature.

### Example 3

Stripless Depilatory Composition 3.

Ingredient. (1) Glyceryl Hydrogenated Rosinate 60.0 (2) Paraffin 30.0 (3) Beeswax 10.0. Procedure. Mix all components and heat at 90 to 95C. to a clear liquid. Cool to room temperature.

### Example 4

Stripless Depilatory Composition 4.

Ingredients. (1) Glyceryl Hydrogenated Rosinate 60.0 (2) Polypropylene Glycol 10.0 (3) Paraffin 23.0 (4) Beeswax 7.0. Procedure. Mix all components and heat at 90 to 95C. to a clear liquid. Cool to room temperature.

### Example 5

Method of Application.

Hair should be at least ¼ inches long in order to work properly. Heat the product in a microwave or hot water till it turns into a flowable liquid. With an appropriate applicator, spread it in an even, thin, layer onto skin where depilation is desired. Allow the product to harden. It transforms into a uniform strip or film. With a quick, but nice and even motion, remove this strip, pulling in the opposite direction of hair growth. Repeat above steps in other areas of skin.

### Example 6

Consumer Testing of Compositions of Example 1, 2, 3, 4, and 13.

A six-person panel, all female, tested the compositions of example 1 to example 4 by following the application procedure in Example 5. The overall preference ratings, based on ease of product application, completeness of hair removal in a single treatment, ease of cleanup after use, and amount of skin irritation, from most preferred to least preferred, follow: Example 1>Example 4>>>Example 3>Example 2>>>Example 13. Two commercially purchased products (Brazilian wax products mentioned herein), were also tested as controls. All Examples, from 1 to 4, were preferred over the above two commercially purchased Brazilian wax products, and also Example 13 (composition made according to U.S. Pat. No. 5,1 54,919; Garets) when tested by the same group of panelists. These tests were conducted all at the same time.

### Example 7

Stripless Depilatory Composition 5.

Ingredients. (1) Glyceryl Hydrogenated Rosinate 72.0 (2) Polypropylene Glycol 5.0 (3) Paraffin 16.0 (4) Polyvinyl Acetate 7.0. Procedure. Mix (1) to (4) and heat at 90 to 95C. till a clear solution is obtained. Cool to room temperature.

### Example 8.

Stripless Depilatory Composition 6.

Ingredients. (1) Glyceryl Hydrogenated Rosinate 50.0 (2) Paraffin 20.0 (3) Glyceryl Dibehenate 10.0 (4) Polyamide-3 20.0. Procedure. Mix (1) to (4) and heat at 90 to 95C. till a clear solution is obtained. Cool to room temperature.

### Example 9

Stripless Depilatory Composition 7.

Ingredients. (1) Phenol/Styrene/Methylstyrene Copolymer 60.0 (2) Polypropylene Glycol 5.0 (3) Paraffin 35.0. Procedure. Mix (1) to (3) and heat at 90 to 95C. till a clear solution is obtained. Cool to room temperature.

### Example 10

Stripless Depilatory Composition 8.

Ingredients. (1) Glyceryl Hydrogenated Rosinate 53.0 (2) Ethylenediamine/Hydrogenated Dimer Dilinoleate Copolymer Bis-Di-C14-18 Alkyl Amide 20.0 (3) Paraffin 20.0 (4) Beeswax 7.0. Procedure. Mix (1) to (4) and heat at 90 to 95C. till a clear solution is obtained. Cool to room temperature.

### Example 11

Stripless Depilatory Composition 9.

Ingredients. (1) Glyceryl Hydrogenated Rosinate 73.0 (2) Triethyl citrate 20.0 (3) Polyvinyl acetate 7.0. Procedure. Mix (1) to (3) and heat at 90 to 95C. till a clear solution is obtained. Cool to room temperature.

### Example 12

Stripless Depilatory Composition 10.

Ingredients. (1) Glyceryl Hydrogenated Rosinate 60.0 (2) Triethyl citrate 20.0 (3) Polyvinyl acetate 20.0. Procedure. Mix (1) to (3) and heat at 90 to 95C. till a clear solution is obtained. Cool to room temperature. This composition can also be coated on pre-formed strips for pre-coated strips markets.

### Example 13

Stripless Composition 11.

A composition according to U.S. Pat. No. 5,154,919 (Garets) was prepared.

### Example 14

Comparison of Various Depilatory Systems.

The table in FIG. 1 summarizes the advantages of stripless depilatory compositions disclosed herein compared to various other commonly used depilatory compositions, as tested and commented by a test panel of 10 people.

FIG. 1.

The compositions disclosed in the present invention can also be delivered to skin surface via various devices, for example, U.S. patent application Ser. No. 20030004522 (Spina et al.), U.S. Pat. No. 5,847,363 (Debourg et al.); U.S. Pat. No. 6,726,390 (van Steenbergen); and U.S. Pat. No. 4,860,688 (Nazzarro), among others.

### BRIEF DESCRIPTION OF THE DRAWINGS

FIG. 1 Comparison of Depilatory Systems.

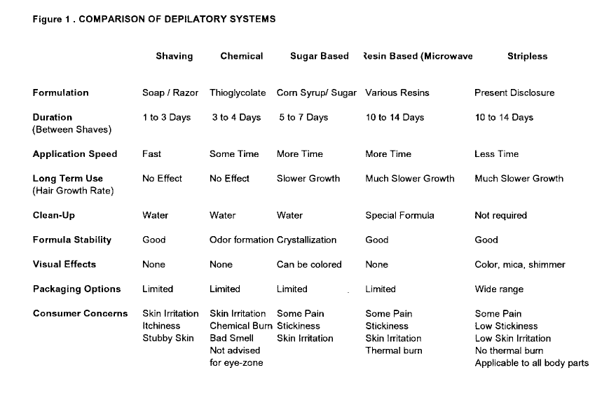

What is claimed is:

1. A depilatory composition that can be applied in liquid form on the surface of the skin, and which is removable by peeling away from the surface of the skin after setting to a pliable film, said composition consisting of (i) a pliable film forming agent, (ii) a hair binding agent, and (iii) a hydroxylic matrix forming agent, and, wherein the relative amounts of said pliable film forming agent, said hair binding agent, and said hydroxylic matrix forming agent are such that said composition upon first heating to a liquid state, then applying to skin transforms to a pliable film, which is capable of catching hairs without adhering to the skin and being removable by peeling away from the skin along with the hairs caught in the pliable film.

2. A depilatory composition according to claim 1, wherein pliable film-forming agent is from 20.0 to 50.0 by weight %.

3. A depilatory composition according to claim 1, wherein hair-binding agent is from 50.0 to 80.0 by weight %.

4. A depilatory composition according to claim 1, wherein hydroxylic matrix forming agent is from 1.0 to 20.0 by weight %.

5. A depilatory composition according to claim 1, wherein said pliable film forming agent is polyvinyl acetate.

6. A depilatory composition according to claim 1, wherein said pliable film forming agent is polyethylene.

7. A depilatory composition according to claim 1, wherein said pliable film forming agent is paraffin wax.

8. A depilatory composition according to claim 1, wherein said pliable film forming agent is beeswax.

9. A depilatory composition according to claim 1, wherein said hair binding agent is a natural resin.

10. A depilatory composition according to claim 1, wherein said hair binding agent is a modified resin.

11. A depilatory composition according to claim 1, wherein said hair binding agent is a synthetic resin.

12. A depilatory composition according to claim 1, wherein said hair binding agent is Phenol/Styrene/Methylstyrene Copolymer.

13. A depilatory composition according to claim 1, wherein said hair binding agent is Polyamide-3.

14. A depilatory composition according to claim 1, wherein said hair binding agent is Ethylenediamine/Hydrogenated Dimer Dilinoleate Copolymer Bis-Di-C14-18 Alkyl Amide.

15. A depilatory composition according to claim 1, wherein said hair binding agent is Glyceryl Hydrogenated Rosinate.

16. A depilatory composition according to claim 1, wherein said hair binding agent is Polyvinyl Acetate.

17. A depilatory composition according to claim 1, wherein said hydroxylic matrix forming agent is polypropylene glycol.

18. A depilatory composition according to claim 1, wherein said hydroxylic matrix forming agent is polyethylene glycol.

19. A depilatory composition according to claim 1, wherein said hydroxylic matrix forming agent is selected from ethylene glycol, propylene glycol, butylene glycol, methylpropanediol, hexylene glycol, and combinations thereof.

20. A depilatory composition according to claim 1, wherein said hydroxylic matrix forming agent is selected from Triethyl citrate, Trimethyl citrate, Tripropyl citrate, Tributyl citrate, Butyl Trihexyl citrate, Acetyl Triethyl Citrate, Acetyl-tri-n-butyl Citrate, Acetyl tri-n-hexyl Citrate, n-Butyl tri-n-hexyl Citrate, and combinations thereof.

21. A depilatory composition according to claim 1, wherein said hydroxylic matrix forming agent is Triethyl citrate.

22. A depilatory composition according to claim 1, wherein said hydroxylic matrix forming agent is selected from Methyl lactate, Ethyl lactate, Propyl lactate, Isopropyl lactate, Butyl lactate, Benzyl lactate, and combinations thereof.

23. A depilatory composition according to claim 1, wherein a filler, selected from calcium carbonate, magnesium sulfate, sodium chloride, Zeolite, Montmorillonite, starch, flour, magnesium aluminum silicate, silica, fumed silica, polyethylene bead, polyethylene powder, mica, ground seeds, ground shells, ground nut shells, and clay is included.

24. A composition according to claim 1, wherein additional ingredients, selected from emollients, moisturizers, skin surface cleansing agents, botanical extracts, skin soothing agents, antibacterial agents, anti-wrinkle additives, anti-aging compositions, skin smoothing agents, UV absorbers, perfumes, colorants, preservatives, color stabilizers, mica pigments, antioxidants, and combinations thereof, are also included.

25. A method for removing hair which comprises applying to the surface of skin containing hair to be removed a disposable depilatory composition consisting essentially of (i) a pliable film forming agent, (ii) a hair binding agent, and (iii) a hydroxylic matrix forming agent, and, wherein the relative amounts of said pliable film forming agent, said hair binding agent, and said hydroxylic solubilizing are such that said composition upon first heating to a liquid state, then applying to skin transforms to a pliable film, which is capable of catching hairs without adhering to the skin and being removable by peeling away from the skin along with the hairs caught in the pliable film.

26. The method according to claim 25, which further comprises heating device or delivery system prior to applying it to the surface of skin.

27. The method according to claim 25, which further comprises use of a strip to ease the peeling away of the pliable film from the skin along with the hairs caught in the pliable film.

28. The method according to claim 25, wherein the depilatory composition can be pre-coated on a fabric, non-woven fabric, plastic, paper, or compressed cellulose strip, which can then be applied to skin for hair removal thus avoiding the pre-heating step.

29. The method according to claim 28, wherein the strip is selected from fabric, non-woven fabric, plastic, paper, compressed cellulose, or combinations thereof.
